(19) United States
(12) Patent Application Publication
(10) Pub. No.: US 2018/0085004 A1
(43) Pub. Date: Mar. 29, 2018

(54) SYSTEM AND METHOD FOR DIAGNOSING PIGMENTED LESION

(71) Applicant: SPECLIPSE, INC., Seoul (KR)

(72) Inventors: Sung Hyun PYUN, Seoul (KR); Wan Ki MIN, Gyeonggi-do (KR)

(21) Appl. No.: 15/540,366

(22) PCT Filed: Apr. 4, 2017
(86) PCT No.: PCT/KR2017/003703

§ 371 Date: Jun. 28, 2017

(30) Foreign Application Priority Data
Apr. 4, 2016 (KR) 10-2016-0041316
Apr. 6, 2016 (KR) 10-2016-0041993
Sep. 8, 2016 (KR) 10-2016-0115675
Jan. 26, 2017 (KR) 10-2017-0012935

(51) Int. Cl. A61B 5/00; A61B 5/103

(52) U.S. Cl. CPC A61B 5/0095; A61B 5/444; A61B 5/1032; A61B 5/7278

(57) ABSTRACT

According to one embodiment, a system for diagnosing a pigmented lesion is provided. The system includes: a sound detector configured to detect a photoacoustic wave which is generated when a first laser is irradiated onto a target having a pigmented lesion; a disease diagnosis unit configured to determine a type of the pigmented lesion based on generated light which is generated when a second laser is irradiated onto the target; and a pigmented lesion diagnosis unit configured to calculate at least one of a depth and a thickness of the pigmented lesion based on a time at which the photoacoustic wave is detected and laser irradiation time information on the first laser irradiated onto the target.

### FIELD

The present invention relates to a system and a method for diagnosing a pigmented lesion.

The present invention was supported by National Research and Development Project Business as follows:

{National Research and Development Project Business Supporting the Present Invention}

[Project Number] N056300067

[Related Department] Ministry of Trade, Industry and Energy

[Research Management Specialized Agency] Korea Institute for Advancement of Technology

[Research Business Name] 2017 Commercialization Connection Technology Development Business (R&BD) Private Investment Connection Form (Take-off Platform-TOP)

[Research Project Title] Development and Commercialization of Real-Time and Non-invasive Skin Cancer Diagnosis Device Based On Laser-Induced Plasma/Fluorescence Spectroscopy

[Contribution Rate] 1/1

[Main Institute] Speclipse, Inc.

[Research Period] Apr. 1, 2017-Dec. 31, 2018

### BACKGROUND ART

Pigmented lesions are one of skin diseases and may be divided into benign lesions and malignant lesions. For example, the benign lesion may include freckle or nevus of Ota, and the malignant lesion may include a skin cancer.

The benign lesion may be removed by lasers using a skin toning device or a skin peeling device, and the wavelength of a laser to be used varies according to the location of a benign lesion (depth from a skin).

In related-art methods, it is difficult to distinguish between the benign lesion and the malignant lesion. Therefore, many people who engage in removing pigmented lesions with lasers using a skin toning device or a skin peeling device may treat pigmented lesions without exactly distinguishing them. Even if they can distinguish between the benign lesion and the malignant lesion, they should measure the depth of the benign lesion by cutting out the benign lesion or using expensive equipment, such as an Optical Coherence Tomography (OCT), in order to know the depth of the benign lesion. This may cause inconvenience.

### DETAILED DESCRIPTION OF THE PRESENT DISCLOSURE

### Technical Objects

According to one embodiment of the present invention, there is provided a system and a method for diagnosing a pigmented lesion, which can determine a depth and/or thickness of a pigmented lesion.

According to another embodiment of the present invention, there is provided a system and a method for diagnosing a pigmented lesion, which can recommend a type of laser of a wavelength which can remove a benign lesion to a user based on a depth and/or thickness of a pigmented lesion, and can inform the user of a development state and a degree of seriousness of a malignant lesion.

### Technical Solving Means

According to one embodiment of the present invention, there is provided a system for diagnosing a pigmented lesion, the system including: a sound detector configured to detect a photoacoustic wave which is generated when a first laser is irradiated onto a target having a pigmented lesion; a disease diagnosis unit configured to determine a type of the pigmented lesion based on generated light which is generated when a second laser is irradiated onto the target; and a pigmented lesion diagnosis unit configured to calculate at least one of a depth and a thickness of the pigmented lesion based on a time at which the photoacoustic wave is detected and laser irradiation time information on the first laser irradiated onto the target.

According to another embodiment of the present invention, there is provided a method for diagnosing a pigmented lesion, the method including the steps of: irradiating a first laser onto a target having a pigmented lesion; detecting a photoacoustic wave which is generated from the target onto which the first laser is irradiated; calculating a depth and/or thickness of the pigmented lesion based on laser irradiation time information and a sound detection time at which the photoacoustic wave is detected; determining a type of the pigmented lesion by analyzing a spectrum of light which is generated when a second laser is irradiated onto the target; and, when it is determined that the pigmented lesion is a benign lesion as a result of determining the type of the pigmented lesion, selecting a type of a laser corresponding to the depth of the pigmented lesion.

### Advantageous Effect

According to one or more embodiments of the present invention, the system and the method may recommend a type of a laser having a wavelength capable of removing a benign lesion to a user based on a depth and/or thickness of a pigmented lesion. In addition, according to one or more embodiments of the present invention, the system and the method may diagnose whether a pigmented lesion is a benign lesion or a malignant lesion, measure the depth and/or thickness of the pigmented lesion, recommend a type of a laser capable of removing the benign lesion when the pigmented lesion is determined to be the benign lesion, and inform the user of a development state of the malignant lesion when the pigmented lesion is determined to be the malignant lesion.

### BRIEF DESCRIPTION OF THE DRAWINGS

FIG. 1 is a view to illustrate a system for diagnosing a pigmented lesion according to one embodiment of the present invention;

FIG. 2 is a view to illustrate a method for calculating a depth of a pigmented lesion in the pigmented lesion diagnosis system according to one embodiment of the present invention;

FIG. 3 is a view to illustrate a photoacoustic wave couple being generated;

FIG. 4 is a view to illustrate a system for diagnosing a pigmented lesion according to another embodiment of the present invention;

FIG. 5 is a view to illustrate a system for diagnosing a pigmented lesion according to another embodiment of the present invention;

FIG. 6 is a view to illustrate a system for diagnosing a pigmented lesion according to another embodiment of the present invention; and

FIG. 7 is a view to illustrate a method for diagnosing a pigmented lesion according to one embodiment of the present invention.

### EXPLANATION OF SIGNS

1: laser
2: light collector
3: handpiece
4: spectrometer
5: sound detector
7: diagnosis unit
10: diagnosis device
11: pigmented lesion DB
12: laser type DB
13: disease diagnosis reference spectrum DB
14: spectrum data comparison unit
18: image comparison unit
19: light detector

### BEST MODE FOR EMBODYING THE INVENTION

Exemplary embodiments will now be described more fully with reference to the accompanying drawings to clarify aspects, other aspects, features and advantages of the present invention. The exemplary embodiments may, however, be embodied in many different forms and should not be construed as limited to the exemplary embodiments set forth herein. Rather, the exemplary embodiments are provided so that this disclosure will be thorough and complete, and will fully convey the scope of the application to those of ordinary skill in the art.

If the terms such as “first” and “second” are used to describe elements, these elements should not be limited by such terms. These terms are used for the purpose of distinguishing one element from another element only. The exemplary embodiments include their complementary embodiments.

In the description of this application, the term “element A and/or element B” is used to refer to “at least one of element A and element B.”

In the description of this application, when “element A” and “element B” are referred to as being coupled to each other, “element A” and “element B” are directly coupled to each other or indirectly coupled to each other. Herein, indirectly coupling means that there is one or more third elements between “element A” and “element B.”

The terms “unit” and “module” and the terms having suffix “-er” or “-or” used in the description of this application refer to a unit for processing at least one function or operation, and may be implemented by hardware, software, or a combination of hardware and software.

Expressions such as “transmitting,” “communicating,” “receiving,” “providing,” or “forwarding” signals, data, or information, used in the description of this application, or other expressions similar to the aforementioned expressions may refer to directly forwarding signals, data, or information from one element (“element a”) to another element (“element b”), and also refer to forwarding to element b via at least one other element (“element c”).

In the description of this application, elements “operatively related to each other” should be interpreted as being connected with each other in a wired and/or wireless manner so as to transmit and/or receive data.

Although there is no explicit expression “an element (“element a”) and another element (“element b”) are operatively related to each other” in the description, if element a receives signals, data, or information outputted from element b directly or through at least one other element and performs its operation (“element a”), or if element b receives signals, data, or information outputted from element a directly or through at least one other element and performs its operation (“element b”), it should be understood that element a and element b are “operatively related to each other.”

In the description of this application, a “laser generation device” refers to a device which generates a laser for beauty or medical care, and an “aesthetic or medical laser handpiece” refers to a device which has a shape to be held by the user and is configured to receive a laser generated by the laser generation device and emit the laser to a target.

In the description of this application, the term “laser” means a pulse laser or a continuous light laser. In addition, the frequency band of the “laser” may have a certain frequency band, for example, an ultra violet (UV) band, a visible light band, or an infrared (IR) band.

In the description of this application, the term “generated light” encompasses all types of light which are generated when a laser is irradiated onto a target (for example, body tissue). Accordingly, the “generated light” may refer to plasma light, reflected light, scattered light, and/or fluorescent light.

In the description of this application, the term “wavelength” refers to a specific numerical value or a numerical value of a specific range (that is, a wavelength band).

In the description of this application, the term “first laser” refers to a laser which is irradiated onto a target to obtain a photoacoustic wave or a photoacoustic wave couple which is used to calculate a depth and/or thickness of a pigmented lesion.

In the description of this application, the term “second laser” refers to a laser which is irradiated onto a target to obtain generated light for determining a type of a pigmented lesion.

In the description of this application, the term “third laser” refers to a laser which is irradiated onto a target to obtain an image of a pigmented lesion.

In the description of this application, the “first laser,” “second laser,” and “third laser” will be referred to as a “laser” when it is not necessary to distinguish them.

In embodiments of the present invention which will be described with reference to the accompanying drawings, the first laser and the second laser may be the same laser or the first laser and the second laser may be lasers which are irradiated onto a target at different times.

In embodiments of the present invention which will be described with reference to the accompanying drawings, the first laser and the third laser may be the same laser or the first laser and the third laser may be lasers which are irradiated onto a target at different times.

In embodiments of the present invention which will be described with reference to the accompanying drawings, the second laser and the third laser may be the same laser or the second laser and the third laser may be lasers which are irradiated onto a target at different times.

In embodiments of the present invention which will be described with reference to the accompanying drawings, the first laser and the second laser may be the same laser or the first laser and the second laser may be lasers which are irradiated onto a target at different times.

In embodiments of the present invention which will be described with reference to the accompanying drawings, the first laser, the second laser, and the third laser may be the same laser or the first laser, the second laser, and the third laser may be lasers which are irradiated onto a target at different times.

The terms used herein are for the purpose of describing particular exemplary embodiments only and are not intended to limit the present invention. As used herein, the singular forms are intended to include the plural forms as well, unless the context clearly indicates otherwise. It will be further understood that the terms “comprises” and/or “comprising,” when used in this specification, do not preclude the presence or addition of one or more other components.

Hereinafter, the present invention will be described in greater detail with reference to the accompanying drawings. In describing specific embodiments, various specific features are described to assist in a detailed description and a comprehensive understanding of the present invention. However, it is apparent that the exemplary embodiments can be carried out by those of ordinary skill in the art without those specifically defined features. In the description of exemplary embodiments, certain detailed explanations of portions which are well known and have nothing to do with the present invention are omitted when it is deemed that they may unnecessarily obscure the essence of the present invention.

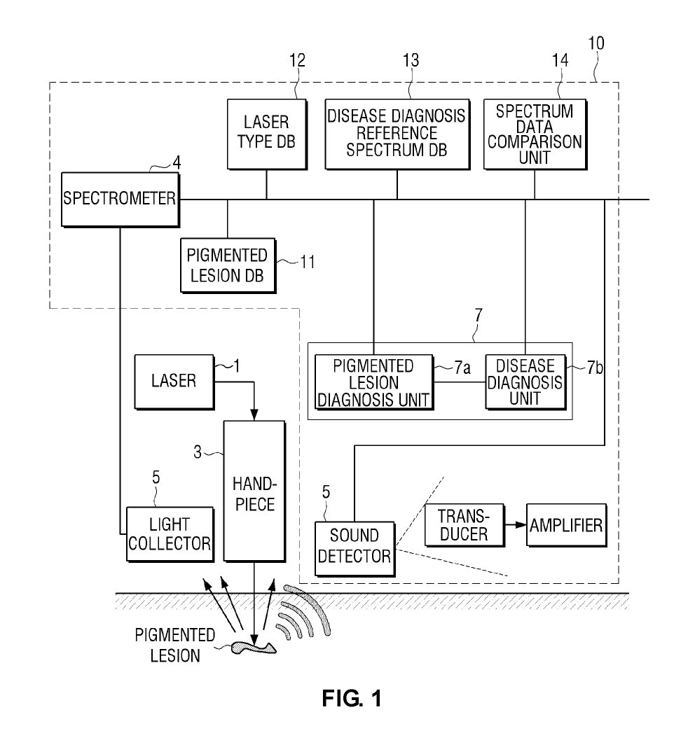

FIG. 1 is a view to illustrate a system for diagnosing a pigmented lesion according to one embodiment of the present invention.

Referring to FIG. 1, the system for diagnosing the pigmented lesion according to one embodiment of the present invention includes a laser generation device 1, a light collector 2, an aesthetic or medical laser handpiece 3 (hereinafter, referred to as a “handpiece”), and a device for diagnosing a pigmented lesion 10 (hereinafter, referred to as a “diagnosis device”).

The diagnosis device 10 includes a spectrometer 4, a sound detector 5, a diagnosis unit 7, a pigmented lesion DB storage unit 11, a laser type DB storage unit 12, a disease diagnosis reference spectrum DB storage unit 13, and a spectrum data comparison unit 14.

The diagnosis unit 7 includes a pigmented lesion diagnosis unit 7a and a disease diagnosis unit 7b.

In the description of this application, a “result of sound detection” may include a “sound detection time” or “data for calculating the sound detection time.”

According to the present embodiment described with reference to FIG. 1, a first laser for obtaining a photoacoustic wave and a second laser for obtaining generated light may be the same laser. Accordingly, in the present embodiment described with reference to FIG. 1, the first laser and the second laser is referred to as a “laser” without being distinguished from each other.

In the present embodiment, the sound detector 5 may detect a sound which is generated when a laser is irradiated onto a target (for example, a skin) from which a depth and/or thickness of a pigmented lesion is to be identified. For example, the sound detector 5 may include a transducer to convert a sound into an electric signal to detect a sound, and an amplifier to amplify the electronic signal converted by the transducer.

When a laser generated by the laser generation device 1 is irradiated onto a target through the handpiece 3, a pigmented lesion existing in the target may absorb the laser and generate a sound. This phenomenon is referred to as a photoacoustic phenomenon, and a sound generated by the photoacoustic phenomenon will be referred to as a “photoacoustic wave” in the following description.

In the present embodiment, the pigmented lesion diagnosis unit 7a may calculate the depth of the pigmented lesion based on a time at which the photoacoustic wave is detected by the sound detector 5 (“sound detection time”), and laser irradiation time information.

In the description of this application, the term “laser irradiation time information” may be used to mean at least one of:

a laser generation time at which the laser generation device generates a laser to be irradiated onto a target;

a time at which the laser reaches the surface of the target;

a time at which light generated from the surface of the target after the laser is irradiated onto the target is detected; and

a certain time between i) the laser generation time and iii) the time at which the generated light is detected.

The above-described times included in the laser irradiation time information may be obtained in various methods, and for example, the laser generation time may be obtained by the laser generation device 1 or a controller (not shown) for controlling a laser generation operation of the laser generation device 1, and the time at which the laser reaches the surface of the target may be obtained by a device (for example, a photodiode) for detecting a laser in the proximity of the surface of the target. The time at which the generated light is detected may be obtained by the light collector 2. At least one of the obtained times is provided to the pigmented lesion diagnosis unit 7a.

The sound detector 5 may detect a photoacoustic wave which is generated from the target when the laser is irradiated onto the target, and may provide the result of sound detection to the pigmented lesion diagnosis unit 7a.

The sound detector 5 may detect one or more photoacoustic waves and the pigmented lesion diagnosis unit 7a may calculate the depth and/or thickness of the pigmented lesion using the sound detection time and the laser irradiation time information.

In the present embodiment, the pigmented lesion diagnosis unit 7a may receive the result of sound detection from the sound detector 5, and may calculate the depth and/or thickness of the pigmented lesion using the result of sound detection and the laser irradiation time information.

The sound detector 5 may detect at least two photoacoustic waves.

In the description of this application, a wave which is detected first when the first laser is irradiated onto the target is referred to as a first photoacoustic wave, and a wave which is detected next is referred to as a second photoacoustic wave. When the first photoacoustic wave and the second photoacoustic wave satisfy the following condition, the first photoacoustic wave and the second photoacoustic wave are referred to as a “photoacoustic wave couple.”

Condition

in which the first photoacoustic wave and the second photoacoustic wave are detected in sequence after the laser is irradiated onto the target, and a time at which the first photoacoustic wave is detected and a time at which the second photoacoustic wave is detected are different from each other, and it is determined that the first photoacoustic wave is generated when the first laser is absorbed onto the top surface of the pigmented lesion (a surface close to the surface of the target) and the second photoacoustic wave is generated when the laser is absorbed onto the bottom surface of the pigmented lesion (a surface far from the surface of the target).

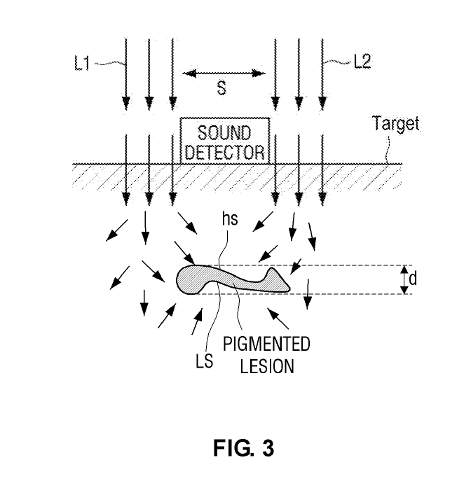

FIG. 3 is a view to illustrate a photoacoustic wave couple being generated.

Referring to FIG. 3, when lasers L1, L2 are irradiated onto a pigmented lesion of a target, the first photoacoustic wave is generated at a time when the lasers are absorbed onto the top surface (hs) of the pigmented lesion, and the second photoacoustic wave is generated when the lasers are absorbed onto the bottom surface (Ls) of the pigmented lesion. Since these photoacoustic waves have a photoacoustic wave couple relationship, the thickness (d) of the pigmented lesion may be calculated.

To generate the second photoacoustic wave at the time when the lasers are absorbed onto the bottom surface (Ls) of the pigmented lesion, it is preferable that the lasers which are spaced from each other by a predetermined distance (S) are irradiated onto the periphery of the pigmented lesion rather than being irradiated onto the center of the pigmented lesion as shown in FIG. 3.

The pigmented lesion diagnosis unit 7a may calculate the thickness of the pigmented lesion based on a result of detecting the photoacoustic wave couple.

The pigmented lesion diagnosis unit 7a may perform an operation of selecting one or more types of leasers for treating the pigmented lesion with reference to the laser type DB when the pigmented lesion is a benign lesion. In the description of this application, the term “laser type” is data defining a laser and this data defines a laser according to at least one of a wavelength, power, a pulse width, and a product name.

Specifically, the pigmented lesion diagnosis unit 7a selects one or more types of lasers corresponding to the depth and/or thickness of the pigmented lesion from the laser type DB.

For example, the pigmented lesion diagnosis unit 7a may select one or more types of lasers in consideration of the depth of the pigmented lesion, or may select one or more types of lasers in consideration of both the depth and the thickness of the pigmented lesion.

This is because the wavelength, power, and width (duration) of the laser for removing or reducing the pigmented lesion vary according to the depth and/or thickness of the pigmented lesion. The pigmented lesion diagnosis unit 7a may determine a development state of the pigmented lesion with reference to the pigmented lesion DB when the pigmented lesion is a malignant lesion. Herein, the malignant lesion may be a skin cancer, for example, and the pigmented lesion DB is data which defines a development state according to the depth and/or thickness of the pigmented lesion.

The wavelength of the laser for treating the pigmented lesion varies according to the location of the pigmented lesion. For example, a Q-switched Ruby laser (694 nm) and a Q-switched Alexandrite laser (755 nm) may treat a pigmented lesion existing on the top of epidermis, and a Q-switched Nd: YAG laser (532 nm) may treat a pigmented lesion existing in epidermis, and a Q-switched Nd: YAG laser (1064 nm) may treat a pigmented lesion existing in corium.

The pigmented lesion diagnosis unit 7a may determine the development state of the pigmented lesion according to the depth and/or thickness of the pigmented lesion by referring to the pigmented lesion DB when the pigmented lesion is a malignant lesion as described above. For example, the pigmented lesion diagnosis unit 7a may determine the development state according to the thickness of the pigmented lesion or may determine the development state in consideration of both the depth and the thickness of the pigmented lesion.

“Data defining a development state” may indicate a development stage of a cancer (first stage, second stage, third stage, fourth stage).

The above-described pigmented lesion DB and the laser type DB may be included in the diagnosis device 10 as shown in FIG. 1, or may be separately stored in an external storage device (not shown) that the pigmented lesion diagnosis unit 7a can access in a wire or wireless manner. When the pigmented lesion DB and the laser type DB are stored in the external storage device, the pigmented lesion diagnosis unit 7a may access the pigmented lesion DB and the laser type DB stored in the external storage device and refer to data.

Although not shown in FIG. 1, a display like a monitor may display a result of an operation of the pigmented lesion diagnosis unit 7a. For example, the type of laser selected by the pigmented lesion diagnosis unit 7a or the development state of the pigmented lesion may be displayed on the display (not shown).

In the above-described embodiment, the pigmented lesion diagnosis unit 7a performs the operation of selecting the type of laser when the pigmented lesion is a benign lesion and performs the operation of determining the development state when the pigmented lesion is a malignant lesion.

The disease diagnosis unit 7b may determine the type of the pigmented lesion.

The disease diagnosis unit 7b may be configured to determine whether the pigmented lesion is a malignant lesion by analyzing a spectrum of light generated from the pigmented lesion when the laser is irradiated onto the pigmented lesion.

When the laser generated by the laser generation device 1 is irradiated onto the target having the pigmented lesion through the handpiece 3, the photoacoustic wave and the generated light are generated.

The photoacoustic wave may be detected by the sound detector 5 and provided to the pigmented lesion diagnosis unit 7a as described above. The light collector 2 collects the generated light and provides the generated light to the spectrometer 4.

The spectrometer 4 measures the spectrum of the generated light. The spectrum data comparison unit 14 compares the spectrum of the generated light which is measured by the spectrometer and the disease diagnosis reference spectrum DB. The disease diagnosis unit 7b determines whether the pigmented lesion is a malignant lesion or a benign lesion based on a result of comparing by the spectrum data comparison unit 14. A result of determining by the disease diagnosis unit 7b is provided to the pigmented lesion diagnosis unit 7a.

When the spectrum of the generated light is the same or very similar as or to the spectrum in the disease diagnosis reference spectrum DB, the disease diagnosis unit 7b may determine that the pigmented lesion is a malignant lesion. Korean Patent Registration No. 10-1640202 (Jul. 11, 2016) discloses technology of determining whether there is a disease using a spectrum of generated light, so please refer to the disclosure of Korean Patent Registration No. 10-1640202 (Jul. 11, 2016). The features disclosed in Korean Patent Registration No. 10-1640202 (Jul. 11, 2016) are incorporated herein as a part of the description of this application.

In the embodiment described with reference to FIG. 1, the first laser and the second laser are the same laser. However, a modification can be made to the embodiment.

According to one modification of FIG. 1, the first laser and the second laser are irradiated onto the target at different times. That is, the sound detector 5 detects a photoacoustic wave when the first laser generated by the laser generation device 1 is irradiated onto the target. The light collector 2 collects light generated from the target when the second laser generated by the laser generation device 1 later than the first laser is irradiated onto the target.

According to another modification of FIG. 1, the second laser is irradiated onto the target earlier than the first laser. That is, when the second laser generated by the laser generation device 1 is irradiated onto the target, the light collector 2 collects light generated from the target. When the first laser generated by the laser generation device 1 later than the second laser is irradiated onto the target, the sound detector 5 detects the photoacoustic wave.

In the present embodiment described with reference to FIG. 1, the light collector 2 is not included in the pigmented lesion diagnosis device 10. However, this is merely an example, and the light collector 2 may be configured to be included in the pigmented lesion diagnosis device 10. In addition, a certain element from among the elements included in the pigmented lesion diagnosis device 10, for example, the sound detector 5, may be configured not to be included in the pigmented lesion diagnosis device 10.

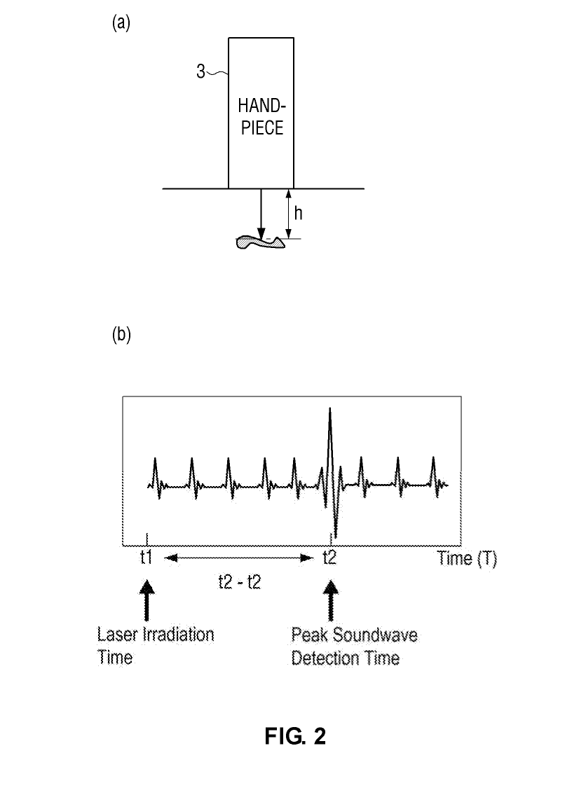

FIG. 2 is a view to illustrate a method for calculating a depth of a pigmented lesion in the pigmented lesion diagnosis system according to one embodiment of the present invention.

In the description of this application, a depth (that is, a location) of a pigmented lesion is defined by a distance (h) from the surface of a target to the pigmented lesion (for example, the surface lesion (top surface, bottom surface or side surface) of the pigmented or a certain location in the pigmented lesion) (see view (a) of FIG. 2). In view (a) of FIG. 2, the depth (h) of the pigmented lesion is illustrated by a distance to the center of the pigmented lesion, but this is merely an example.

For example, the pigmented lesion diagnosis unit 7a may calculate the depth of the pigmented lesion based on following Equation 1:

Depth (h) of a pigmented lesion=speed of a photoacoustic wave×(sound detection time−laser irradiation time) Equation 1

The “laser irradiation time” included in Equation 1 refers to one of:

a laser generation time at which the laser generation device generates a laser to be irradiated onto a target;

a time at which the laser reaches the surface of the target;

a time at which light generated from the surface of the target after the laser is irradiated onto the target is detected; and

a certain time between the laser generation time and the time at which the generated light is detected.

Herein, the sound detection time refers to a time at which a photoacoustic wave generated from the pigmented lesion is detected. When the laser is irradiated onto the pigmented lesion, a photoacoustic wave having a relatively higher peak than in the other portions may be generated from the pigmented lesion as shown in view (b) of FIG. 2.

In addition, the pigmented lesion diagnosis unit 7a may calculate the thickness of the pigmented lesion based on following equation 2:

Thickness (d) of a pigmented lesion=speed of a photoacoustic wave×(second photoacoustic wave detection time-first photoacoustic wave detection time) Equation 2

Herein, the first photoacoustic wave and the second photoacoustic wave are a photoacoustic wave couple.

In the present embodiment, the entirety or part of the elements included in the pigmented lesion diagnosis device 10 may be configured to be coupled to the handpiece 3. For example, the sound detector 5 and/or the light collector 2 may be coupled to a handpiece detachable device (not shown in FIG. 1), and the handpiece detachable device may be attachably and detachably coupled to the handpiece 3. In another example, the sound detector 5 and/or the light collector 2 may be mounted in the handpiece 3.

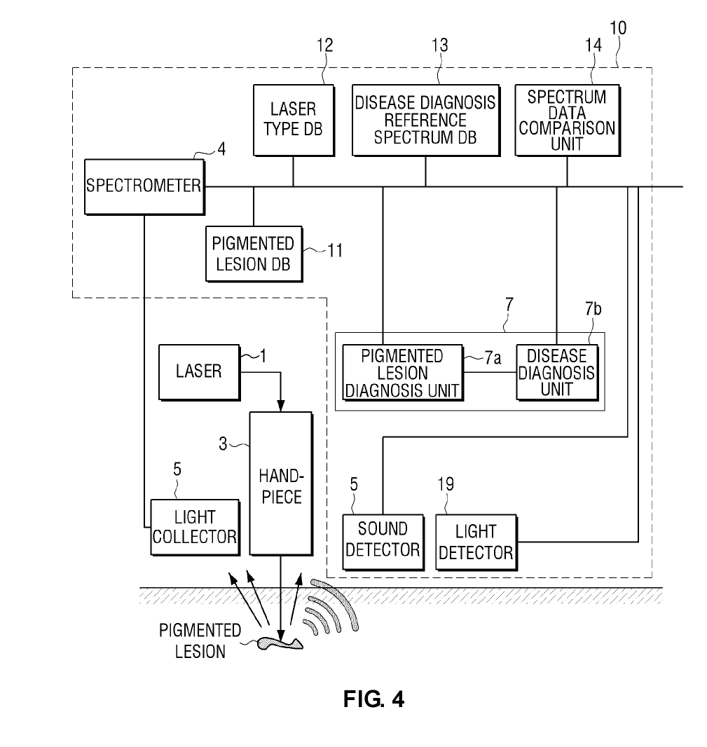

FIG. 4 is a view to illustrate a system for diagnosing a pigmented lesion according to another embodiment of the present invention.

Referring to FIG. 4, the pigmented lesion diagnosis system according to one embodiment of the present invention includes a laser generation device 1, a light collector 2, an aesthetic or medical laser handpiece 3, and a pigmented lesion diagnosis device 10.

The diagnosis device 10 includes a spectrometer 4, a sound detector 5, a diagnosis unit 7, a pigmented lesion DB storage unit 11, a laser type DB storage unit 12, a disease diagnosis reference spectrum DB storage unit 13, a spectrum data comparison unit 14, and a light detector 19.

The diagnosis unit 7 includes a pigmented lesion diagnosis unit 7a and a disease diagnosis unit 7b.

From among the elements of the present embodiment described with reference to FIG. 4 and the elements of the embodiment described with reference to FIG. 1, the elements given the same reference numerals perform the same or similar functions.

The present embodiment described with reference to FIG. 4 differs from the embodiment described with reference to FIG. 1 in that the light detector 19 obtains laser irradiation time information and provides it to the pigmented lesion diagnosis unit 7a. Hereinafter, the difference of the embodiment of FIG. 4 from the embodiment of FIG. 1 will be mainly described.

According to the present embodiment described with reference to FIG. 4, a first laser for obtaining a photoacoustic wave and a second laser for obtaining generated light are the same laser. Accordingly, in the embodiment described with reference to FIG. 4, the first laser and the second laser is referred to as a “laser” without being distinguished from each other.

In the present embodiment, the sound detector 5 may detect a photoacoustic wave which is generated when a laser is irradiated onto a target having a pigmented lesion.

The light detector 19 may detect the laser which generates the photoacoustic wave detected by the sound detector 5 or generated light which is generated from the target after the laser is irradiated onto the target.

The light detector 19 may be implemented by using a device like a photo diode, for example. The light detector 19 may be configured to detect a laser which travels through the inside of the handpiece 3, detect a laser which is outputted from the handpiece 3 and travels to the target, or generated light which is generated from the target.

When the light detector 19 is configured to detect the laser traveling through the inside of the handpiece 3, the light detector 19 may be coupled to the inside of the handpiece 3 or the outside of the handpiece 3.

When the light detector 19 is configured to detect the laser traveling through the inside of the handpiece 3, an optical element (not shown in FIG. 4) may be disposed inside the handpiece 3 to divide at least a part of the laser traveling through the inside of the handpiece 3 toward the light detector 19.

When the light detector 19 is configured to detect the laser which is outputted from the handpiece 3 and travels to the target or detect the generated light generated from the target, the light detector 19 may be disposed adjacent to a certain portion of the handpiece 3 (a portion contacting the target) or may be disposed in a handpiece detachable device (not shown in FIG. 4) which is attachably and detachably coupled to the handpiece 3.

The pigmented lesion diagnosis unit 7a may calculate the depth and/or thickness of the pigmented lesion based on a time at which the photoacoustic wave is detected by the sound detector 5 and a result of laser detection obtained by the light detector 19. Herein, the “result of laser detection” includes “laser irradiation time information.”

The pigmented lesion diagnosis unit 7a may select a type of laser and/or a development state based on the depth and/or thickness of the pigmented lesion.

Regarding elements which have not been described from among the elements of the present embodiment described with reference to FIG. 4, please refer to the embodiments described with reference to FIGS. 1 to 3.

In the embodiment described with reference to FIG. 4, the first laser and the second laser are the same laser. However, a modification can be made to the embodiment.

According to one modification of FIG. 4, the first laser and the second laser are irradiated onto the target at different times. That is, the sound detector 5 detects a photoacoustic wave when the first laser generated by the laser generation device 1 is irradiated onto the target. The light collector 2 collects light generated from the target when the second laser generated by the laser generation device 1 later than the first laser is irradiated onto the target. According to another modification of FIG. 4, the second laser is irradiated onto the target earlier than the first laser. That is, the light collector 2 collects light generated from the target when the second laser generated by the laser generation device 1 is irradiated onto the target. The sound detector 5 detects a photoacoustic wave when the first laser generated by the laser generation device 1 later than the second laser is irradiated onto the target.

In the above-described modifications, the light detector 19 may be configured to detect the light generated from the target by the first laser or the first laser.

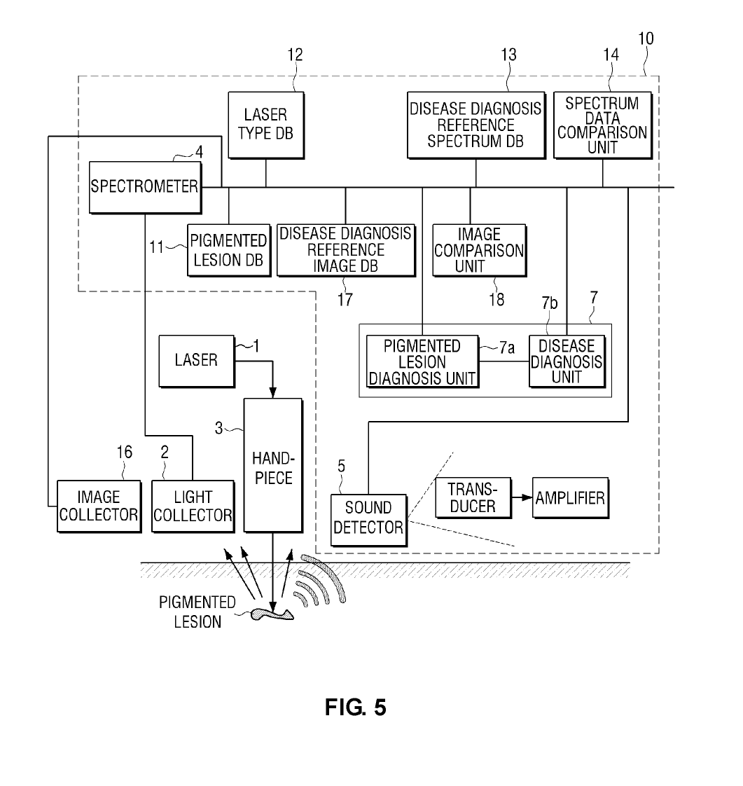

FIG. 5 is a view to illustrate a system for diagnosing a pigmented lesion according to another embodiment of the present invention.

Referring to FIG. 5, the pigmented lesion diagnosis system according to another embodiment of the present invention includes a laser generation device 1, a light collector 2, an aesthetic or medical laser handpiece 3, a pigmented lesion diagnosis device 10, and an image collector 16.

The diagnosis device 10 includes a spectrometer 4, a sound detector 5, a diagnosis unit 7, a pigmented lesion DB storage unit 11, a laser type DB storage unit 12, a disease diagnosis reference spectrum DB storage unit 13, a spectrum data comparison unit 14, a disease diagnosis reference image DB storage unit 17, an image comparison unit 18, and a light detector 19.

The diagnosis unit 7 includes a pigmented lesion diagnosis unit 7a and a disease diagnosis unit 7b.

From among the elements of the present embodiment described with reference to FIG. 5 and the elements of the embodiment described with reference to FIG. 1, the elements given the same reference numerals perform the same or similar functions.

According to the present embodiment described with reference to FIG. 5, a first laser for obtaining a photoacoustic wave, a second laser for obtaining generated light, and a third laser for obtaining an image of a pigmented lesion are the same laser. Accordingly, in the present embodiment described with reference to FIG. 5, the first laser, the second laser, and the third laser are referred to as a “laser” without being distinguished from one another.

The embodiment described with reference to FIG. 5 differs from the embodiment described with reference to FIG. 1 in that the disease diagnosis unit 7b determines a disease in consideration of both the spectrum of generated light and the image of the pigmented lesion. Hereinafter, the difference of the embodiment of FIG. 5 from the embodiment of FIG. 1 will be mainly described.

In the present embodiment, the sound detector 5 may detect a photoacoustic wave which is generated when a laser is irradiated onto a target from which a depth and/or thickness of a pigmented lesion is to be identified.

The pigmented lesion diagnosis unit 7a may calculate the depth and/or thickness of the pigmented lesion based on a time at which the photoacoustic wave is detected by the sound detector 5 and laser irradiation time information, and may determine a type of laser and/or a development state.

The image collector 16 may collect an image regarding the target when the laser is irradiated onto the target or may collect an image regarding the target when the laser is not irradiated onto the target.

The image comparison unit 18 compares the image collected by the image collector 16 and the disease diagnosis reference image DB.

The spectrum data comparison unit 14 compares the spectrum of the generated light measured by the spectrometer 4 and the disease diagnosis reference spectrum DB.

A result of comparing by the image comparison unit 18 and a result of comparing by the spectrum data comparison unit 14 are provided to the disease diagnosis unit 7b, and the disease diagnosis unit 7b determines a type of the pigmented lesion based on the result of comparing the images and the result of comparing the spectrum data.

The disease diagnosis unit 7b determines the type of the pigmented lesion by reflecting at least one of the result of comparing by the spectrum data comparison unit 14 and the result of comparing by the image comparison unit 18.

According to one example of a determination method, the disease diagnosis unit 7b determines that the pigmented lesion is a malignant lesion i) when there is the same or very similar data as or to the spectrum data of the generated light in the disease diagnosis reference spectrum data DB, or ii) when there is the same or very similar image as or to the image of the pigmented lesion in the disease diagnosis reference image DB.

According to another example of a determination method, the disease diagnosis unit 4b determines that the pigmented lesion is a malignant lesion i) when there is the same or very similar data as or to the spectrum data of the generated light in the disease diagnosis reference spectrum data DB, and ii) when there is the same or very similar image as or to the image of the pigmented lesion in the disease diagnosis reference image DB.

Korean Patent Registration No. 10-1640202 (Jul. 11, 2016) discloses technology of determining whether a pigmented lesion is a malignant lesion by analyzing a spectrum of light generated from the pigmented lesion and/or an image of the pigmented lesion. The entirety of the features disclosed in Korean Patent Registration No. 10-1640202 (Jul. 11, 2016) are incorporated herein as a part of the description of this application.

In the embodiment described above with reference to FIG. 5, the first laser, the second laser, and the third laser are the same laser, but a modification can be made to the embodiment.

According to one modification of FIG. 5, lasers are irradiated onto the target two times at different times.

From among the lasers irradiated two times, the laser irradiated to the target first is the first laser and the laser irradiated next is the second laser and the third laser.

Alternatively, from among the lasers irradiated two times, the laser irradiated onto the target first is the first laser and the third laser and the laser irradiated next is the second laser.

Alternatively, from among the lasers irradiated two times, the laser irradiated onto the target first is the second laser and the laser irradiated next is the first laser and the third laser.

Alternatively, from among the lasers irradiated two times, the laser irradiated onto the target first is the second laser and the third laser and the laser irradiated next is the first laser.

According to another modification of FIG. 5, lasers are irradiated onto the target three times at different times.

From among the lasers irradiated three times, the laser irradiated onto the target first is the first laser, the laser irradiated next is the second laser, and the laser finally irradiated is the third laser.

Alternatively, from among the lasers irradiated three times, the laser irradiated onto the target first is the second laser, the laser irradiated next is the second laser, and the laser finally irradiated is the second laser.

Alternatively, from among the lasers irradiated three times, the laser irradiated onto the target first is the third laser, the laser irradiated next is the first laser, and the laser finally irradiated is the second laser.

Regarding elements which have not been described from among the elements of the present embodiment described with reference to FIG. 5, please refer to the embodiments described with reference to FIGS. 1 to 3.

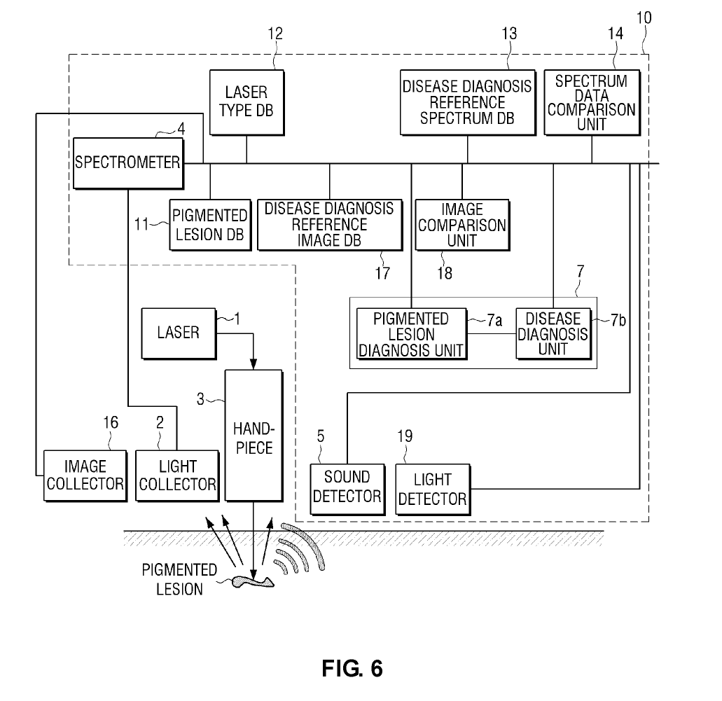

FIG. 6 is a view to illustrate a system for diagnosing a pigmented lesion according to another embodiment of the present invention.

Referring to FIG. 6, the pigmented lesion diagnosis system according to another embodiment of the present invention includes a laser generation device 1, a light collector 2, an aesthetic or medical laser handpiece 3, a pigmented lesion diagnosis device 10, and an image comparison unit 18.

The diagnosis device 10 includes a spectrometer 4, a sound detector 5, a diagnosis unit 7, a pigmented lesion DB storage unit 11, a laser type DB storage unit 12, a disease diagnosis reference spectrum DB storage unit 13, a spectrum data comparison unit 14, an image collector 16, a disease diagnosis reference image DB storage unit 17, and a light detector 19.

The diagnosis unit 7 includes a pigmented lesion diagnosis unit 7a and a disease diagnosis unit 7b.

From among the elements of the present embodiment described with reference to FIG. 6 and the elements of the embodiment described with reference to FIG. 1, the elements given the same reference numerals perform the same or similar functions.

According to the present embodiment described with reference to FIG. 6, a first laser for obtaining a photoacoustic wave, a second laser for obtaining generated light, and a third laser for obtaining an image of a pigmented lesion are the same laser. Accordingly, in the present embodiment described with reference to FIG. 6, the first laser, the second laser, and the third laser are referred to as a “laser” without being distinguished from one another.

The embodiment described with reference to FIG. 6 differs from the embodiment described with reference to FIG. 1 in that the light detector 19 obtains laser irradiation time information and provides it to the pigmented lesion diagnosis unit 7a, and the disease diagnosis unit 7b determines a type of a disease in consideration of both the spectrum of generated light and an image of the pigmented lesion.

Hereinafter, the difference of the embodiment of FIG. 6 from the embodiment of FIG. 1 will be mainly described.

In the present embodiment, the sound detector 5 may detect a photoacoustic wave which is generated when a laser is irradiated onto a target from which a depth and/or thickness of a pigmented lesion is to be identified.

The pigmented lesion diagnosis unit 7a may calculate the depth and/or thickness of the pigmented lesion based on a time at which the photoacoustic wave is detected by the sound detector 5 and laser irradiation time information, and may determine a type of laser and/or a development state.

Regarding the operations of the image collector 16 and the image comparison unit 18, please refer to the embodiment described with reference to FIG. 5.

A result of comparing by the image comparison unit 18 and a result of comparing by the spectrum data comparison unit 14 are provided to the disease diagnosis unit 7b, and the disease diagnosis unit 7b determines a type of the pigmented lesion based on the result of comparing the images and the result of comparing the spectrum data.

The disease diagnosis unit 7b determines the type of the pigmented lesion by reflecting at least one of the result of comparing by the spectrum data comparison unit 14 and the result of comparing by the image comparison unit 18.

Regarding examples of a determination method by reflecting the result of comparing the images and the result of comparing the spectrum data by the disease diagnosis unit 7b, please refer to the embodiment described with reference to FIG. 5.

The pigmented lesion diagnosis unit 7a may calculate the depth and/or thickness of the pigmented lesion based on the time at which the photoacoustic wave is detected by the sound detector 5 and a result of laser detection obtained by the light detector 19. Regarding the operation of the light detector 19, please refer to the embodiment described with reference to FIG. 4.

Regarding elements which have not been described from among the elements of the present embodiment described with reference to FIG. 6, please refer to the embodiments described with reference to FIGS. 1 to 3.

According to one modification of FIG. 6, lasers are irradiated onto the target two times at different times.

From among the lasers irradiated two times, the laser irradiated to the target first is the first laser and the laser irradiated next is the second laser and the third laser.

Alternatively, from among the lasers irradiated two times, the laser irradiated onto the target first is the first laser and the third laser and the laser irradiated next is the second laser.

Alternatively, from among the lasers irradiated two times, the laser irradiated onto the target first is the second laser and the laser irradiated next is the first laser and the third laser.

Alternatively, from among the lasers irradiated two times, the laser irradiated onto the target first is the second laser and the third laser and the laser irradiated next is the first laser.

According to another modification of FIG. 6, lasers are irradiated onto the target three times at different times.

From among the lasers irradiated three times, the laser irradiated onto the target first is the first laser, the laser irradiated next is the second laser, and the laser finally irradiated is the third laser.

Alternatively, from among the lasers irradiated three times, the laser irradiated onto the target first is the second laser, the laser irradiated next is the second laser, and the laser finally irradiated is the second laser.

Alternatively, from among the lasers irradiated three times, the laser irradiated onto the target first is the third laser, the laser irradiated next is the first laser, and the laser finally irradiated is the second laser.

In the above-described modifications, the light detector 19 may detect the first laser irradiated onto the target or detect light generated when the first laser is irradiated onto the target.

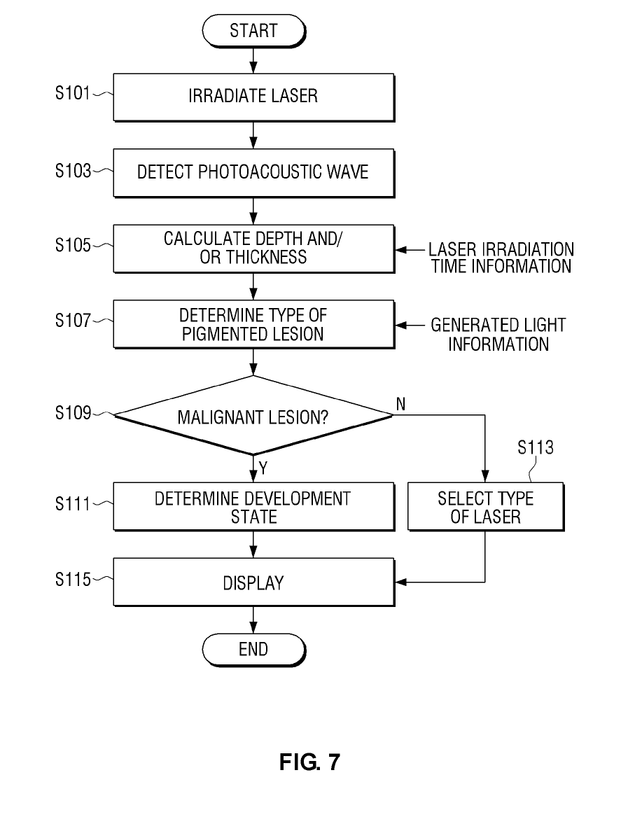

FIG. 7 is a view to illustrate a method for diagnosing a pigmented lesion according to one embodiment of the present invention.

Referring to FIG. 7, the method for diagnosing the pigmented lesion according to one embodiment of the present invention includes: a step (S101) of irradiating a laser (“first laser”) onto a target having a pigmented lesion; a step (S103) of detecting a photoacoustic wave generated from the target onto which the first laser is irradiated; a step (S105) of calculating a depth and/or thickness of the pigmented lesion based on laser irradiation time information and a sound detection time at which the photoacoustic wave is detected; a step (S107) of determining a type of the pigmented lesion by analyzing a spectrum of light which is generated when a laser (“second laser”) is irradiated onto the target; a step (S111) of, when it is determined that the pigmented lesion is a malignant lesion in step S107 (S109: YES), determining a development state (development stage) of the malignant lesion; a step (S113) of, when it is determined that the pigmented lesion is a benign lesion in step S107 (S109: NO), selecting a type of a laser; and a step (S115) of displaying the results of steps S111 and S113 for the user.

The method for determining the pigmented lesion described with reference to FIG. 6 may be implemented by the embodiments of the device for diagnosing the pigmented lesion described with reference to FIGS. 1, 2, 3, 4, 5, and/or 6.

For example, in step S105, the pigmented lesion diagnosis unit 9a may calculate the depth of the pigmented lesion using Equation 1 described above, and may calculate the depth of the pigmented lesion using Equation 2 described above.

In another example, the step (S103) of detecting the photoacoustic wave may be performed by the sound detector 5. Specifically, the step (S103) of detecting a sound may be performed by the sound detector 5 which is coupled to the handpiece 3 which receives a laser and emits the laser to the target.

In the present embodiment, step S107 may be configured to determine the type of the pigmented lesion by analyzing the spectrum of the generated light (for example, the embodiments of FIGS. 1 and 4), or may be configured to determine the type of the pigmented lesion by analyzing the spectrum of the generated light and the image of the pigmented lesion (for example, the embodiments of FIGS. 5 and 6).

In the present embodiment, the method may further include a light detection step of detecting the laser irradiated onto the target. A result of detecting in the light detection step may be used in step S105. The light detection step may be performed by the light detector 19 which detects the laser irradiated onto the target.

In the present embodiment, the method may further include a step of controlling a wavelength, power, and/or a pulse length of the laser generation device which generates a laser to be irradiated onto the target. For example, the step of controlling may be performed by a controller (not shown) which controls the laser generation device to generate a laser having a wavelength, power, and/or a pulse width corresponding to the depth and/or thickness of the pigmented lesion.

In the present embodiment, the first laser and the second laser may be the same laser or may be lasers which are irradiated onto the target at different times.

While the invention has been shown and described with reference to certain preferred embodiments thereof and the drawings, the present invention is not limited to the above-described embodiments, and various modifications or other embodiments which belong to the equivalents of the scope of the present invention can be achieved by a person skilled in the art.

1. A system for diagnosing a pigmented lesion, the system comprising:
a sound detector configured to detect a photoacoustic wave which is generated when a first laser is irradiated onto a target having a pigmented lesion;
a disease diagnosis unit configured to determine a type of the pigmented lesion based on generated light which is generated when a second laser is irradiated onto the target; and
a pigmented lesion diagnosis unit configured to calculate at least one of a depth and a thickness of the pigmented lesion based on a time at which the photoacoustic wave is detected and laser irradiation time information on the first laser irradiated onto the target.

2. The system of claim 1, wherein the pigmented lesion diagnosis unit is further configured to calculate the thickness of the pigmented lesion based on times at which a photoacoustic wave couple is detected and the laser irradiation time information on the first laser.

3. The system of claim 1, wherein the pigmented lesion diagnosis unit is further configured to perform an operation of selecting one or more types of lasers for treating the pigmented lesion from a laser type DB in which at least one laser type matches a depth of a pigmented lesion, and
wherein the operation of selecting is an operation of selecting one or more types of lasers corresponding to the depth of the pigmented lesion from the laser type DB.

4. The system of claim 1, further comprising:
a spectrometer configured to measure a spectrum of the generated light which is generated when the second laser is irradiated onto the target; and
a spectrum data comparison unit configured to compare the spectrum measured by the spectrometer and a disease diagnosis reference spectrum DB,
wherein the disease diagnosis unit is configured to determine the type of the pigmented lesion based on a result of comparing by the spectrum data comparison unit.

5. The system of claim 4, wherein a result of determining by the disease diagnosis unit is provided to the pigmented lesion diagnosis unit, and
wherein, when it is determined that the type of the pigmented lesion is a malignant lesion, the pigmented lesion diagnosis unit is configured to determine a development state of the pigmented lesion.

6. The system of claim 1, wherein the first laser and the second laser are the same laser.

7. The system of claim 1, wherein the first laser and the second laser are irradiated onto the target at different times.

8. The system of claim 1, further comprising a light detector configured to detect the first laser irradiated onto the target or detect the generated light which is generated when the first laser is irradiated onto the target,
wherein the laser irradiation time information comprises a time at which the light detector detects.

9. The system of claim 4, further comprising:
an image collector configured to collect an image of the target when a third laser is irradiated onto the target; and
an image comparison unit configured to compare a disease diagnosis reference image DB and the image collected by the image collector,
wherein the disease diagnosis unit is configured to determine the type of the pigmented lesion by reflecting at least one of the result of comparing by the spectrum data comparison unit and a result of comparing by the image comparison unit.

10. The system of claim 9, wherein the first laser, the second laser, and the third laser are the same laser,
the first laser and the second laser are the same laser and the third laser is a laser which is irradiated onto the target at a different time from a time at which the first laser is irradiated onto the target,
the first laser and the third laser are the same laser and the second laser is a laser which is irradiated onto the target at a different time from a time at which the first laser is irradiated onto the target, or
the second laser and the third laser are the same laser and the first laser is a laser which is irradiated onto the target at a different time from a time at which the second laser is irradiated onto the target.

11. The system of claim 9, further comprising a light detector configured to detect the first laser irradiated onto the target or detect the generated light which is generated when the first laser is irradiated onto the target,
wherein the laser irradiation time information comprises a time at which the light detector detects.

12. The system of claim 1, wherein the laser irradiation time information comprises at least one of:
i) a laser generation time at which a laser generation device generates the first laser to be irradiated onto the target;
ii) a time at which the first laser reaches a surface of the target;
iii) a time at which the light generated from the surface of the target after the first laser is irradiated onto the target is detected; and
iv) a certain time between the time at which the first laser is generated and the time at which the generated light is detected.

13. A method for diagnosing a pigmented lesion, the method comprising the steps of:
irradiating a first laser onto a target having a pigmented lesion;
detecting a photoacoustic wave which is generated from the target onto which the first laser is irradiated;
calculating a depth and/or thickness of the pigmented lesion based on laser irradiation time information and a sound detection time at which the photoacoustic wave is detected;
determining a type of the pigmented lesion by analyzing a spectrum of light which is generated when a second laser is irradiated onto the target; and
when it is determined that the pigmented lesion is a benign lesion as a result of determining the type of the pigmented lesion, selecting a type of a laser corresponding to the depth of the pigmented lesion.

14. The method of claim 13, further comprising, when it is determined that the pigmented lesion is a malignant lesion as a result of determining the type of the pigmented lesion, determining a development state of the pigmented lesion.

15. The method of claim 14, further comprising a light detection step of detecting the first laser or generated light which is generated when the first laser is irradiated onto the target, and
wherein the calculating of the depth and/or thickness of the pigmented lesion comprises calculating the depth and/or thickness of the pigmented lesion based on the time at which the photoacoustic wave is detected and a time at which the first laser or the generated light is detected as a result of performing the light detection step.

16. The method of claim 14, wherein the laser irradiation time information comprises at least one of:
i) a laser generation time at which a laser generation device generates the first laser to be irradiated onto the target;
ii) a time at which the first laser reaches a surface of the target;
iii) a time at which the light generated from the surface of the target after the first laser is irradiated onto the target is detected; and
iv) a certain time between the time at which the first laser is generated and the time at which the generated light is detected.

17. The method of claim 14, wherein the first laser and the second laser are the same laser.

18. The method of claim 14, wherein the first laser and the second laser are lasers which are irradiated onto the target at different times.
